(19) United States
(12) Patent Application Publication
(10) Pub. No.: US 2010/0003734 A1
(43) Pub. Date: Jan. 7, 2010

(54) Production of recombinant photosynthetic bacteria which produces molecular hydrogen in a light independent manner and hydrogen evolution method using above strain

(76) Inventors: Mi-Sun Kim, Daejeon (KR); You-Kwan Oh, Daejeon (KR); Jeong K. Lee, Seoul (KR); Eui-Jin Kim, Seoul (KR)

Correspondence Address: MCNAIR LAW FIRM, P.A., P.O. BOX 447, GREENVILLE, SC, 29602-0447, US

(21) Appl. No.: 12/316,686

(22) Filed: Dec. 16, 2008

(30) Foreign Application Priority Data
Jul. 3, 2008 (KR) 10-2008-0064177

(51) Int. Cl. C12P 3/00; C12N 1/21; C12N 15/66

(52) U.S. Cl. 435/168; 435/252.3; 435/471

(57) ABSTRACT

A method of making a photosynthetic bacteria variant which that can produce hydrogen in both day and night. In one embodiment hydrogen is produced in a light independent manner by adding pyruvate lyase and formate lyase complex to a photosynthetic strain of Rhodobacter sphaeroides. A preferred photosynthetic bacteria variant that can produce hydrogen in both day and night from Rhodobacter sphaeroides is KCTC 12085.

### FIELD OF THE INVENTION

This invention relates generally to hydrogen production of a variant from Rhodobacter sphaeroides which expresses foreign enzyme related to anaerobic Hydrogen production. Conventional Rhodobacter sphaeroides wild type strain is capable of producing hydrogen of high efficiency according to the action of nitrogenase under photosynthetic condition, but it has a limitation in producing hydrogen at night where there is no light. In case of ameliorated variant provided in this invention, even at night without light it is possible for anaerobic hydrogen synthase which is expressed after being introduced from the outside to produce hydrogen, and in case of variant from the above Rhodobacter sphaeroides, it is possible for anaerobic hydrogen synthase which was introduced together with conventional nitrogenase to function simultaneously under photosynthetic condition. This has much more improved hydrogen productivity.

More specifically, this invention relates to production of recombinant photosynthetic bacteria which produces molecular hydrogen in a light independent manner and hydrogen evolution method using above strain. Technically, this invention allows recombinant photosynthetic bacteria especially a variant of Rhodobacter sphaeroides expressing foreign enzyme related to anaerobic hydrogen production to produce hydrogen according to the anaerobic hydrogen synthase even at night with no light. In photosynthetic condition, this manufacturing method of recombinant photosynthetic bacteria allows anaerobic hydrogen synthase to function with nitrogenase simultaneously and it is about hydrogen evolution method using above strain.

### BACKGROUND

Limited fossil fuel use on earth is developing various environmental problems as well as financial problems. In this kind of situation, the use of sun light comes under the ultimate technology which humankind has to depend on. The technology using light energy can be possible by various methods. Among them, photosynthesis reserves high light energy efficiency at present after evolution process of 3.5 billion years. Hydrogen emitted from metabolic activity of photosynthetic bacterial strain is clarifying energy and it is said to be the ultimate alternative energy source of the future. Compared to the fossil fuel or atomic power of the present, Hydrogen only produces infinitesimal quantity of pollutant during combustion. Moreover, Hydrogen can be stored as gas or liquid and it has a large potential with a wide range of use.

Photosynthetic bacteria can be classified as purple non-sulfur bacteria, purple sulfur bacteria, green non-sulfur bacteria and green sulfur bacteria. These bacteria have a characteristics of not producing oxygen during the process unlike the photosynthesis of algae and plants. Microorganism, a genus of Rhodobacter which is purple non-sulfur bacteria can grow in various metabolic conditions such as aerobic condition, anaerobic dark condition and anaerobic light condition. What's more, microorganism, the genus of Rhodobacter, has been used as the main subject of technical development of alternative medicine which uses microorganism.

Through the past 50 years of the research, about microorganism, the genus of Rhodobacter, gene manipulation is established with ease to be so similar with E. coli and the genes of the constitutent factors of hydrogen producing nitrogenase and hydrogen synthase are known relatively precisely. Therefore achieving these genes and changing the process of the expression and activation synthetically can maximize the efficiency of hydrogen production. In addition, the genome sequencing was finished in both photosynthetic bacteria which are the subjects of the research, and the information can be used for strain development technique through genome transformation.

Hydrogen development by a genus microorganism of Rhodobacter has been tried in technically developed countries in Europe including Japan, but the direction of the research has a focus on the promotion of hydrogen production through culture process. This approach showed significant improvement in hydrogen production but there was a limit. Now hydrogen productivity has been raised through genetic improvement of the proteins related to nitrogenase and hydrogen synthase which mediates hydrogen production and then the limit can be overcome. Recently, researchers are trying to maximize the hydrogen productivity by understanding the features of hydrogen synthase and by controlling them. The object is to overcome the low production efficiency which is the delaying factor of commercialization of hydrogen energy, the clarifying energy and it will prepare the foundation to rapidly advance the actualization of hydrogen energy use.

Hydrogen production of above photosynthetic strain is the result of proton, H⁺ fixation according to the nitrogenase and at that time the consumed energy totally depends on light reaction. In case of Rhodospirillum rubrum, which is a type of purple non-sulphur bacteria, fermentation using organic acid under anaerobic condition occurs and the fact that hydrogen is produced in this process is widely reported. However, the mechanism of hydrogen production is not precisely investigated and yet there is no progress in the research related to hydrogen production due to the enzyme's complexity and oxygen's sensitivity.

Therefore, technical development for improved hydrogen production using photosynthetic bacteria has been proceeded as a method to optimize the light reaction instead of improving nitrogenase and hydrogen synthase as well as expression control. Thus, through the process of optimizing culture condition for culture strain to receive maximum light, much more energy has been induced to flow as hydrogen production. However, even in this kind of approach, there is a limit which does not overcome the light energy utilizing ability of the light device. Therefore, planning metabolic engineering which induces much more energy to flow to metabolism related to hydrogen production and investigating the culture conditions, etc. to induce activation and stabilization of hydrogen synthase are the subjects in the research to overcome the limitation.

Hydrogenase are functionally classified as hydrogen absorbing enzyme, hydrogen producing enzyme and mediating enzyme acting on both hydrogen absorbing and producing enzymes. According to the cofactor which enzyme has, it is mainly classified into NiFe-Hydrogenase (Appel et al. 2000. Arch Microbiol. 173: 333-338) and Fe-only Hydrogenase (Pan et al. 2003. J. Biol. Inorg. Chem. 8: 469-474). Among them, hydrogen producing enzyme belongs to Fe-only Hydrogen Hydrogenase. NiFe-Hydrogenase mediates both the absorption and the production of hydrogen and improvement of hydrogen production through cloning shows inadequate efficiency. Even in the case of hydrogenase which is concerned with hydrogen production by forming a complex with Formate-Hydrogen Lyase (FHL), it is known as NiFe-Hydrogenase but the precise mechanism of the function is still not revealed.

On the other hand, Rhodobacter sphaeroides KCTC 12085 is a photosynthetic strain separated from the nature and it has a high hydrogen productivity and a high resistance to salt. Since the molecular genetic approach is easy, it has an advantage that manufacturing a variant which promotes hydrogen production is possible. The above strain is similar to other hydrogen producing photosynthetic strains which have been reported as the wild types in the past and it has higher hydrogen producing ability. It has been reported after the observation that when the variants are manufactured by destroying various genes, hydrogen production efficiency has raised more than 3 times. (Lee et al. 2002. Appl. Microbiol. Biotechnol. 60: 147-153).

In early stage of development of above mentioned photosynthetic strain, synthetic light was used to measure the hydrogen production efficiency but in a practical stage sun light must ultimately be used. For above strain, there is a problem in that hydrogen production is impossible at night when there is no light.

This originates from the necessary requirement for light energy in the case of hydrogen production by nitrogenase in above strain. Therefore, it is difficult to expect satisfactory efficiency from hydrogen producing ability of the photosynthetic strain.

Accordingly, a principal object of this invention is to solve the above described problem.

It is another object to manufacture a Rhodobacter sphaeroides variant which is an improved photosynthetic strain producing hydrogen in anaerobic condition at night with no light as well as the daytime with light. For the manufacture, in the genes coding above pyruvate lyase and formate lyase complex protein are recombined and conjugated on Rhodobacter sphaeroides for expression. The purpose is to provide variants within the above manufacturing methods;

Yet another object of this invention is to provide hydrogen production method in culture condition such as creating media which can allow Rhodobacter sphaeroides with the complex of above pyruvate lyase and formate lyase to produce hydrogen and by light fermentation.

### SUMMARY OF THE INVENTION

The invention to achieve the above objects has the following features:

This invention has the important and novel feature of producing hydrogen within a light independent manner by adding pyruvate lyase and formate lyase complex to photosynthetic strain Rhodobacter sphaeroides.

Presently, the Rhodobacter sphaeroides is KCTC 120 8 5 and the pyruvate lyase and formate lyase complex is extracted from Rhodospirillum rubrum.

Moreover, in this invention, to manufacture photosynthetic variant, there were several stages; a stage that partial DNA fragment was obtained at the periphery of pyruvate lyase and formate lyase complex gene in Rhodospirillum rubrum and the recombinant vector was recombined homologously on the chromosome of Rhodospirillum rubrum. a stage which secures pyruvate lyase and formate lyase complex related area from the chromosome of the above recombinant strain, a stage to manufacture one recombinant vector which conjugates above pyruvate lyase and formate lyase complex related area, a stage selecting transformed Rhodobacter sphaeroides after transforming it by conjugating the above recombinant vector from E. coli S17-1 to Rhodobacter sphaeroides.

To achieve another object of this invention, during the hydrogen producing process using photosynthetic strain, in basic composition of hydrogen sistrom minimal medium to cultivate a Rhodobacter sphaeroides variant, ammonium phophomolybdate was substituted with sodium phophomolybdate of same quantity and succinic acid was substituted with glucose for hydrogen production efficiency. Nickel Chloride (NiCl2), sodium selenite (Na2SeO3) and sodium tungstate (Na2VVO4) are added for application.

Improved strain of this invention was capable of producing hydrogen with newly introduced hydrogenase in a condition with no light and we could observe hydrogen production in both conventional nitrogenase and foreign hydrogenase in a condition with light. Comparing with the conventional wild type strain, there was an approximately twice more of hydrogen production efficiency. Hydrogen production method by introducing photosynthetic strain of foreign hydrogenase has not been reported till now and it is the first case to be reported in the world.

Hydrogen production method by using microorganism is much useful when it runs with environmental friendly management of the waste. It is not just a production method of hydrogen energy but also a technology to reduce CO₂ production. To actualize the technology, it is required to develop new strains which produce much hydrogen with resistance to various environmental factors. And these highly efficient strains are the core factor to build up hydrogen production facility and to produce hydrogen energy through the facility.

Furthermore, photosynthetic microorganism is used as the feed for birds and fish at present and produces useful metabolites as well. It is anticipated that the financial value can be maximized by connecting with secondary process which secures remnant biomass and metabolites after hydrogen production.

The foregoing described invention will be better understood by reference to the drawings and detailed description which follows:

### DESCRIPTION OF THE DRAWINGS

A preferred construction designed to carry out the invention will hereinafter be described in detail with reference to the schematic representations provided in the drawings.

FIG. 1 is a graph showing arrangement structure of the genes related to pyruvate lyase and formate lyase complex, which was applied to this invention.

FIG. 2 is a graph with hydrogen production and growth curves of a Rhodobacter sphaeroides strain in aerobic fermentation condition without light.

FIG. 3 is a graph showing hydrogen production and growth curves of a Rhodobacter sphaeroides strain in photosynthetic condition with light.mass and metabolites after hydrogen production.

### DETAILED DESCRIPTION

Through the analytic result of base sequence of Rhodobacter sphaeroides, we could find out the fact that this strain has all the enzymes necessary for the degradation process into pyruvate by using glucose and therefore glucose and other organic acid can be transformed into pyruvate. However, the above strain does not have genes which synthesize pyruvate lyase and formate lyase complex and hydrogen production process according to the fermentation is deleted. Therefore, these genes were obtained from other strains to express in the above strain and to raise hydrogen producing ability much higher.

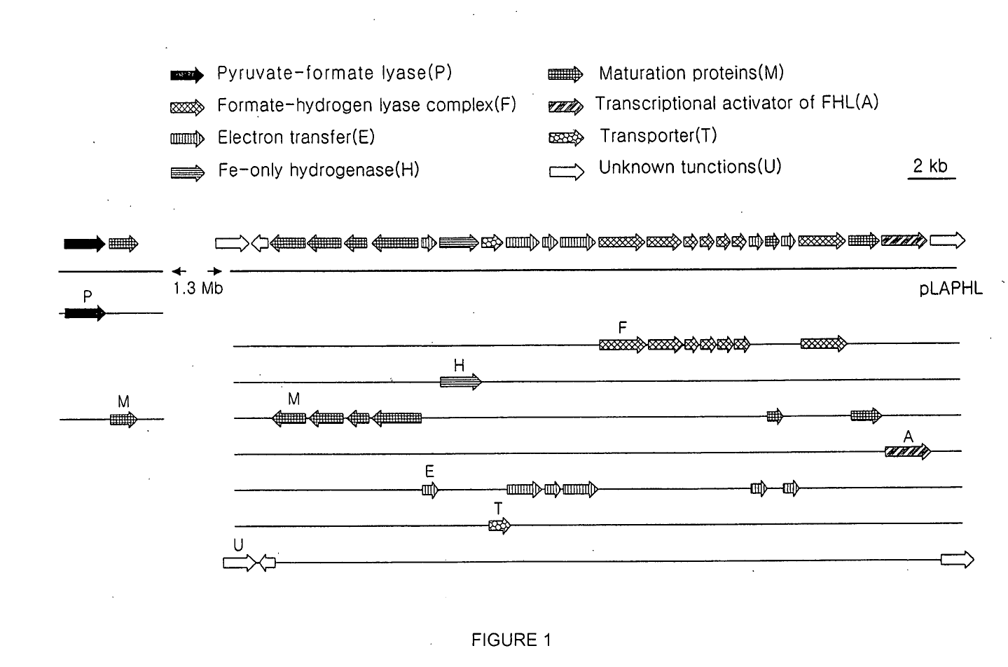

To achieve the genes encoding above pyruvate lyase and formate lyase complex and the areas which include activating factor and transcript regulator related to the activation of these enzymes, a part of DNA in the concerned area was achieved by Polymerase Chain Reaction; PCR. Later, whole gene was obtained by inducing homologous recombination by using the concerned gene fragment and it was cloned to pLA2917 which is the cosmid vector maintaining a large quantity of genes (referred to FIG. 1). pLAPHL Plasmid which was manufactured in this kind of method contains total 25 genes and it was maintained smoothly during mobilization after moving to Rhodobacter sphaeroides.

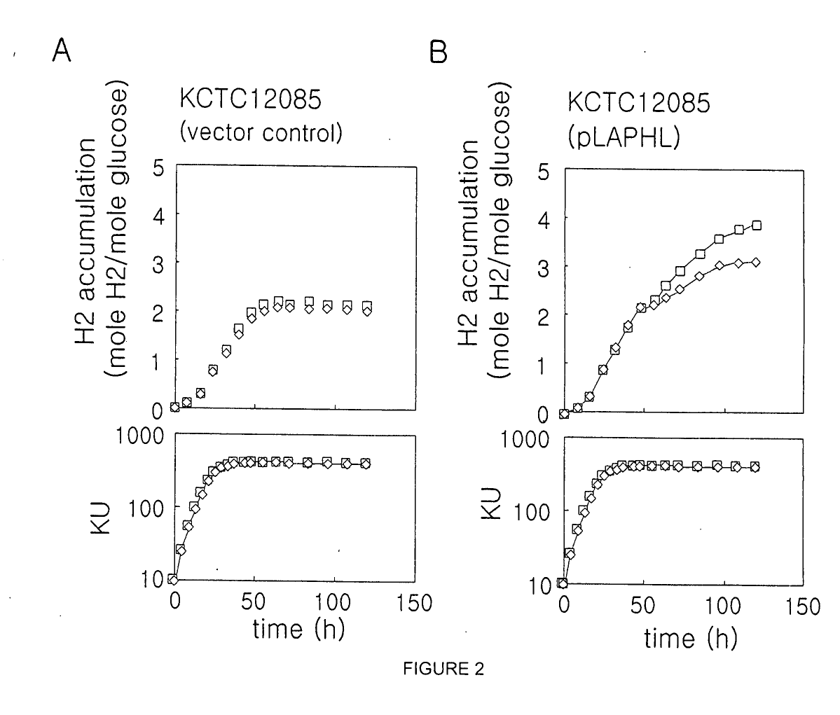

In this invention, a variant including above plasmid and a wild type strain were brought on in anaerobic condition without light to test the hydrogen producing ability (referred to FIG. 2). As the result, the variant showed the growth of cells even in anaerobic condition without light or other final electron acceptors and we could see the production of hydrogen. The result showed 0.4 mole amount of hydrogen production and it is about 20% of 2 mole which is a theoretical production quantity through metabolic process of pyruvate lyase and formate lyase complex from one glucose molecule.

Furthermore, when hypophosphite, the specific inhibitor on pyruvate lyase, was made to be 2 mM, both cell growth and hydrogen production were not completed. Therefore, under above condition, it is possible to infer that hydrogen was produced during the degradation process of pyruvate into formate by pyruvate lyase and this formate into carbon dioxide and hydrogen.

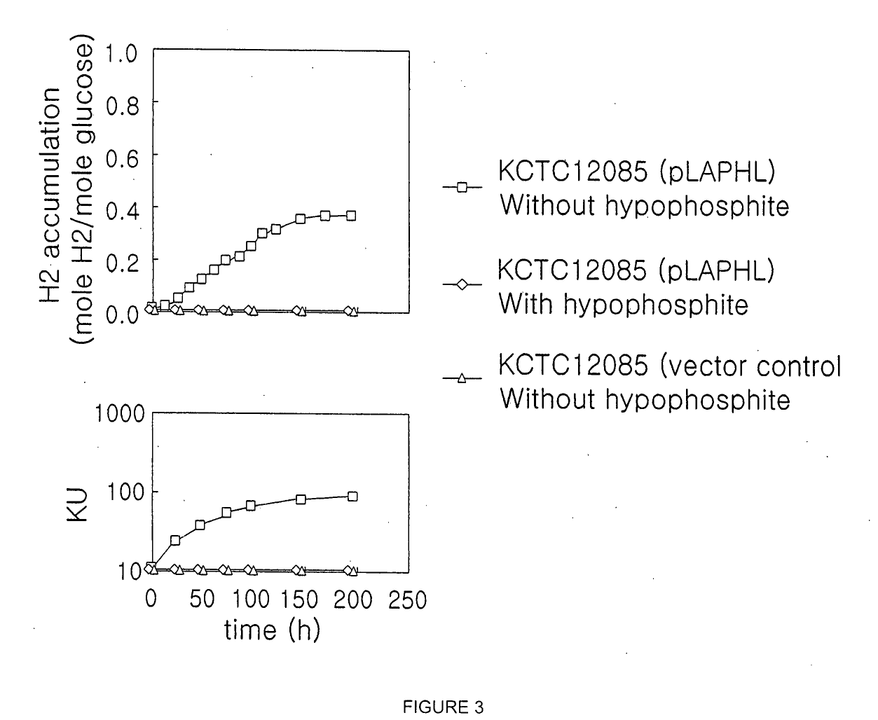

Anaerobic fermentation was still observed in photosynthetic growth condition with given light. When the same strain was raised in photosynthetic growth condition and produced hydrogen was measured, 2 additional moles of hydrogen were produced compared to the production of 2 moles by nitrogenase which was generally observed, making total amount of 4 moles (refer red to FIG. 3). On the other hand, the wild type strain containing vector itself without other genes produced about 2 moles of hydrogen by nitrogenase (referred to FIG. 3). Consequently, there was no difference in the cell growth of the above variant but the production of hydrogen was doubled (refer to FIG. 3).

Management of hypophosphite reduced about 1 mole of hydrogen production and it was still observed when the concentration of the reagent was additionally increased. Thus, about 1 mole of hydrogen is presumed to be formed by pyruvate lyase and formate lyase complex. Remaining 1 mole of hydrogen functions free from pyruvate lyase but it is seen that it is by Fe-Hydrogenase which is another hydrogenase existing on above recombinant vector.

According to the previously reported result, this enzyme showed hydrogen production activity only in the condition with light. Therefore, we can think that there is no effect in the anaerobic fermentation condition without light in FIG. 2 (Km et aL 2008. Int. J. Hydrogen Energy 33: 15t-1521). As the result of this invention, above variant make all the hydrogen production possible by nitrogenase, Fe-hydrogenase and pyruvate lyase, showing much higher hydrogen productivity.

Below, this invention is described very precisely with executed examples but all the executed examples are to indicate this invention is not limited to the content of this specification.

### EXECUTED EXAMPLE 1

### Manufacture of Hydrogen Production Variant Derived from Rhodobacter sphaeroides which Produces Hydrogen in Both Light and Dark Conditions

Whole chromosome DNA of Rhodobacter sphaeroides was separated and approximately 10 kb base sequence including N-terminal region of pyruvate lyase was synthesized in vitro through polymerase chain reaction by using the chromosome as the template. After that, with this region, about 2.-kb transcription and translation terminal sequence which includes streptomycin and spectinomycin resistant gene was cloned with pL01, the suicide vector. Restriction endonuclease and ligase concerned for DNA transection and conjugation were used. We used kanamycin resistant gene which exists on pL01 vector and cloned structures were selected inside E coil after using kanamycin, streptomycin and spectinomycin, with concentration of 25, 50, 50 pg/ml each. Completed structure induced single crossover on the chromosome of Rhodobacter sphaeroides through the homologous recombination method. k was selected after using kanamycin with concentration of 10 pg/ml. Later, the chromosome of the recombined strain was separated, transected by restriction enzymes, Xho\ and XbaI, conjugated on Sal\ and XbaI region of pBluescript (SK-) vector and selected by using streptomycin and spectinomycin for cloning. It was transected again with XbaI and Kpn\ for conjugation on the restriction enzyme region same as cosmid vector 11)+2917. After that pLAP1 was manufactured with 4.4 kb region including genes coding pyruvate lyase and activation proteins.

With similar method, regions with genes related to formate lyase were secured. With the chromosome of Rhodospirillum rubrum as the circular model, about 1.1 kb of DNA was synthesized from about 15 kb upper region of 7 gene coherent region which constitutes formate lyase complex by polymerase chain reaction and it was cloned on pL01 By using this structure, single crossover was induced by homologous recombination on the chromosome phase of Rhodospirillum rubrum. The chromosome of this recombinant strain was separated and transected with KonI and about 29.4 kb region was conjugated with KonI area of above pLAP1 to manufacture pLAPFL. (FIG. 1). Above recombinant vector went through transformation in E coil S17-1 and then it was inserted by conjugation method mentioned below in Rhodobacter sphaeroides. E. coli cells with vector were mixed with the object host cells and they were placed on plate media for 6 to 12 hours of conjugation. Then they are smeared on sistrom limiting plate media with addition of concerned antibiotics to achieve transformed host cells. E. coli S17-1 is auxotroph which does not synthesize proline among amino acid. Therefore they do not grow in limiting plate media without proline and non-transformed host cells cannot grow in the media with 1 pg/ml of antibiotics tetracycline. So transformed host cells with antibiotic resistance can be achieved.

FIG. 1 shows the arrangement structure of the genes related to pyruvate lyase and formate lyase complex. Functional classification mentioned above was done with the foundation of known functions from homeodomain of high similarity with each gene. And each 4.4 kb and 29.4 kb chromosomal region was cloned in once vector.

### EXECUTED EXAMPLE 2

### Measurement of Hydrogen Production by using Above Variant

For hydrogen production Rhodobacter sphaeroides KCTC 12085 was used for the object strain and for the growth, composition of sistrom minimal media [20 mM monobasic potassium phosphate (KH₂PO₄), 3.8 mM ammonium sulfate ((NH₄)₂SO₄), 34 mM succinyl acid, 0.59 mM L-glutamate, 0.30 mM L-asparate, 8.5 mM sodium chloride, 105 mM nitrilotriacetic acid, 12 mM magnesium chloride (MgC1₂6H₂O), 0.23 mM calcium chloride (CaC1₂7H₂O), 25 pM ferrous sulfate (FeSO₄7H₂O), 0.16 μM ammonium molybdate ((NH₄)6Mo₇O₂₄4H₂O), 4.7 μM EDTA, 38 μM zinc sulfate (ZnSO₄7H₂O), 9.1 μM manganese sulfate (MnSO₄H₂O), 16 μM copper sulfate (CuSO₄5H₂O), 0.85 μM cobalt nitrate (II)(Co(NO₃)26H₂O), 18 μM boric acid (H₃BO₃), 8.1 μM nicotinic acid, 15 μM thiamine chloride, 41 nM biotin (Sistrom, W. R, /962. J. Gen. Microbiol. 28: 607-6t) was used for the fundamental composition. Here, to optimize the activity of nitrogenase, ammonium molybdate was substituted with a same amount of sodium molybdate. For hydrogen production efficiency in anaerobic fermentation condition, succinyl acid was substituted with 30 mM of glucose. Nickel Chloride (NiC1₂), sodium selenite (Na₂SeO₃), and sodium tungstate (Na₂VVO₄) each with amount of 10 μM were added.

A variant including above plasmid and a wild type strain were brought on in anaerobic condition without light to test the hydrogen production ability. To measure the production quantity of hydrogen, cells were put inside a sealed bottle designed not to let the air out for the growth in a incubator without light. According to the time, a part of gas phase was drawn out by a sealed syringe designed not to let the air out and it was analyzed by Gas Chromatography; GC, Shimadzu.

FIG. 2 shows hydrogen production and growth curves of Rhodobacter sphaeroides strain in anaerobic fermentation condition with no light. The term 02917 identifies a vector itself and pLAPHL is a recombinant vector including pyruvate lyase and formate lyase complex. We tested the case of adding 2 mM of hypophosphite which is the inhibitor of pyruvate lyase and the case of not adding it.

Furthermore, even in the photosynthetic growth condition with light, we tested a variant with above plasmid and a wild type strain for the ability to produce hydrogen and the growth. Both strains were observed by growing them in a incubator with 10 Watts/m₂ of light. The concentration of hypophosphite was raised to 10 mM and we tested the hydrogen producing ability. However, we could not observe the difference with the case which was managed with 2 mM of above reagent. Produced hydrogen was measured with the same method which was given in a condition without light.

FIG. 3 showed hydrogen production and growth curves of Rhodobacter sphaeroides in photosynthetic condition with light. The term plA2917 identifies a vector itself, and pLAPHL is a recombinant vector which includes pyruvate lyase and formate lyase complex. We experimented a case with addition of 2 mM of hypophosphite which is pyruvate lyase and a case without adding it.

For summary, in this invention it did not depend on light reaction and formate lyase which produces hydrogen from pyruvate-formate lyase, the enzyme forming formate from pyruvate in the fermentation process, and formate was achieved from Rhodospirillum rubrum to introduce to Rhodobacter sphaeroides KCTC 12085 which is the domestic photosynthetic bacteria. So, even at night without light, improved hydrogen productivity was accomplished through production of genetically transformed strain which produces hydrogen from organic acid such as glucose and pyruvate. Till now, it is reported that only Rhodospirilium rubrum among photosynthetic strains can grow by fermenting with the use of pyruvate in anaerobic dark condition and in this process the hydrogen is produced (Gorrell and Uffen./977. J. Bacteriol. 131: 533-543). On the other hand domestic indigenous photosynthetic bacteria, Rhodobacter sphaeroides KCTC 12085 has high hydrogen productivity but it does not hold the gene for growth through fermentation and hydrogen production.

Therefore, in this invention pyrvate lyase and formate lyase complex is achieved from Rhodospirillum rubrum during the fermentation process using pyruvate, and it is introduced inside the domestic indigenous photosynthetic bacteria. So a genetically transformed strain is made to produce hydrogen from organic acid such as glucose and pyruvate even in a dark condition without light, bringing out a result of improved hydrogen productivity.

Our invention is limited only by the scope of the following claims:

1. A method of producing a photosynthetic bacteria variant which can produce hydrogen in both day and night comprising the steps of:
a) providing photosynthetic strain Rhodobacter sphaeroides;
b) adding pyruvate lyase and formate lyase complex to sphaeroides to produce hydrogen independent of light.

2. The method of whereas its Rhodobacter sphaeroides is KCTC 12085.

3. The method of claim 1 wherein said pyruvate lyase and formate lyase complex is extracted from Rhodospirillum rubrum and the photosynthetic bacteria variant can produce hydrogen in both day and night.

4. A method for transforming Rhodobacter sphaeroides for use in producing hydrogen comprising the steps of:
a) obtaining a partial DNA fragment is obtained from the periphery of pyruvate lyase and formate lyase complex gene in Rhodospirilium rubrum and the recombinant vector manufactured through the process is recombined homologously on the chromosome of Rhodospirillum rubrum;
b) securing the region related with pyruvate lyase and formate lyase complex from the chromosome of above recombinant strain;
c) providing one recombinant vector with conjugated region related with above pyruvate lyase and formate lyase complex; and
d) combining the products of steps a), b), and c) and whereby said recombinant vector is transformed through conjugation method from E coil S17-1 to Rhodobacter sphaeroides and the transformed Rhodobacter sphaeroides is screened and photosynthetic bacteria variants which can produce hydrogen in both day and night is produced.

5. A method according to claim 1 wherein a photosynthetic bacteria variant is manufactured to produce hydrogen in both day and night.

6. A method according to claim 4 wherein a photosynthetic bacteria variant is manufactured to produce hydrogen in both day and night.

7. In a hydrogen producing process using a photosynthetic strain to cultivate Rhodobacter sphaeroides variant manufactured according to claim 1 wherein hydrogen sistrom minimal media is the basic composition, the improvement comprising the steps of: substituting ammonium molybdate with an equal quantity of sodium molybdate in the basic composition and, for efficient hydrogen productivity in anaerobic condition, substituting succinyl acid with glucose and Nickel Chloride (NiCl₂), Sodium selenite (Na₂SeO₃), and sodium tungstate thereby producing a hydrogen production method using photosynthetic bacteria variant which can produce hydrogen in both day and night has the above characteristics.

8. A method according to claim 4 wherein a photosynthetic bacteria variant is manufactured to produce hydrogen in both day and night.

9. In a hydrogen producing process using a photosynthetic strain to cultivate Rhodobacter sphaeroides variant manufactured according to claim 4 wherein hydrogen sistrom minimal media is the basic composition, the improvement comprising the steps of: substituting ammonium molybdate with an equal quantity of sodium molybdate in the basic composition and, for efficient hydrogen productivity in anaerobic condition, substituting succinyl acid with glucose and Nickel Chloride (NiCl₂), Sodium selenite (Na₂SeO₃), and sodium tungstate thereby producing a hydrogen production method using photosynthetic bacteria variant which can produce hydrogen in both day and night has the above characteristics.
